(19) United States
(12) Patent Application Publication
(10) Pub. No.: US 2002/0160007 A1
(43) Pub. Date: Oct. 31, 2002

(54) Association of mhc class II allenles and cancer

(76) Inventors: Shiv Pillai, Lexington, MA (US); Cariappa Annaiah, Marlborough, MA (US); David Forcione, West Peabody, MA (US); Subhra Chaudhuri, Madison, CT (US)

Correspondence Address: Janis K Fraser, Fish & Richardson, 225 Franklin Street, Boston, MA, 02110-2804, US

(21) Appl. No.: 10/049,251

(22) PCT Filed: Jun. 11, 2001
(86) PCT No.: PCT/US01/18708

(51) Int. Cl. A61K 39/395; C12Q 1/68

(52) U.S. Cl. 424/155.1; 435/6

(57) ABSTRACT

Particular HLA alleles are found to be associated with human cancer Some HLA alleles are negatively associated with cancer, whereas others show a positive association. This discovery suggests that the immune system plays an important role in the detection and elimination of human tumors. The invention includes methods of determining susceptibility to cancer as well as prognosis. The invention further includes methods of treatment of cancer wherein expression of a particular HLA allele is modulated

### STATEMENT AS TO FEDERALLY SPONSORED RESEARCH

This invention was made with Government support under grant number DAMD 17-97-1-7303 awarded by Department of Defense Breast Cancer Research Program, managed by the U.S. Army Medical Research and Materiel Command. The Government may have certain rights in the invention.

### BACKGROUND

The invention relates to the role of HLA molecules in tumorigenesis and in immune surveillance.

The term “immune surveillance” refers to a natural immunological host resistance to the development of cancer. Several models have been proposed to explain immune surveillance. In virally-induced cancers, e.g. those caused by a viral oncogene, the immune system may recognize a non-self viral protein expressed in a cell of the host and destroy the infected cell. This process thus protects the host from the virally-induced cancer by attacking the initial viral insult.

Several possible mechanisms of immune surveillance have been proposed for non-virally induced cancers. First, immune cells may recognize mutant self proteins, e.g. oncogenes. Because oncogenes generally do not encode true “self” proteins because they may have undergone point mutations or genetic rearrangements, cells expressing oncogenes may theoretically be recognized by the immune system and removed from the body. Second, immune cells may recognize proteins expressed in a highly tissue specific manner, e.g., proteins expressed in a tumor cell that are not normally expressed in the tissue from which the tumor is derived (see, e.g., Coulie et al. (1994) J. Exp. Med. 180:35-42; Brichard et al. (1993) J. Exp. Med. 178:489-495). Third, immune cells may recognize proteins that are poorly expressed during development, but are expressed at high levels in some tumors. These three models attempt to explain the central issue of immune surveillance: mechanisms by which the immune system may detect a tumor cell that lacks a true “non-self” component such as a viral protein.

### SUMMARY

The invention is based, in part, on the discovery that particular HLA alleles are associated with human cancer. Some HLA alleles are negatively associated with cancer, whereas others show a positive association. This discovery suggests that the immune system functions in the detection and elimination of human tumors. The invention includes methods of determining susceptibility to cancer, methods of determining the prognosis of an individual suspected of having cancer, as well as methods of treating cancer by modulating the expression of a particular HLA allele or alleles.

In one aspect, the invention features a method of evaluating an individual's susceptibility to cancer. The method includes the steps of: (1) determining that the individual bears a particular HLA allele; and (2) classifying the individual as having a low susceptibility to cancer based upon the presence of the particular HLA allele. By a “low susceptibility to cancer” is meant a susceptibility to cancer that is less than that of the average person in a population having the same ethnicity and gender as that of the tested individual.

Particularly useful HLA alleles that may be tested in the method of the invention include HLA DQB1*03032 and the HLA DRB1*11 group of alleles. In one embodiment, the method classifies the individual's susceptibility to breast cancer. In a preferred embodiment, the method classifies the individual's susceptibility to early onset breast cancer. Classifying the individual as having a low susceptibility to cancer can be based upon an analysis of either a single HLA allele or multiple HLA alleles, e.g., HLA DQB1*03032 and an HLA DRB1*11 allele, HLA DQB1*03032 and another HLA allele, or an HLA DRB1*11 allele and another HLA allele.

The invention also features a method of evaluating an individual's susceptibility to cancer that includes the steps of: (1) determining that the individual bears a particular HLA allele; and (2) classifying the individual as having a high susceptibility to cancer based upon the presence of the particular HLA allele. By a “high susceptibility to cancer” is meant a susceptibility to cancer that is greater than that of the average person in a population having the same ethnicity and gender as that of the tested individual.

Particularly useful HLA alleles that may be tested in the method of the invention include the HLA DRB3*02 group of alleles. In one embodiment, the method classifies the individual's susceptibility to breast cancer. In a preferred embodiment, the method classifies the individual's susceptibility to early onset breast cancer. Classifying the individual as having a high susceptibility to cancer can be based upon an analysis of either a single HLA DRB3*02 allele, multiple HLA DRB3*02 alleles, or an HLA DRB3*02 allele and other HLA alleles.

The invention also provides a method of evaluating the prognosis of an individual suspected of having cancer. The method includes the steps of: (1) determining that the individual bears a particular HLA allele; and (2) classifying the individual as having a positive prognosis based upon the presence of the particular HLA allele. By a “positive prognosis” is meant a prognosis that is favorable, at least according to one criterion, as compared to that of the average person in a population having the same ethnicity and gender as that of the tested individual.

Particularly useful HLA alleles that may be tested in the method of the invention include HLA DQB1*03032 and the HLA DRB1*11 group of alleles. In one embodiment, the method classifies the prognosis of an individual suspected of having breast cancer. In a preferred embodiment, the method classifies the prognosis of an individual suspected of having early onset breast cancer. Classifying the individual as having a positive prognosis can be based upon an analysis of either a single HLA allele or multiple HLA alleles, e.g., HLA DQB1*03032 and an HLA DRB1*11 allele, HLA DQB1*03032 and another HLA allele, or an HLA DRB1*11 allele and another HLA allele.

The invention also features a method of evaluating the prognosis of an individual suspected of having cancer that includes the steps of: (1) determining that the individual bears a particular HLA allele; and (2) classifying the individual as having a poor prognosis based upon the presence of the HLA allele. By a “poor prognosis” is meant a prognosis that is less favorable, at least according to one criterion, as compared to that of the average person in a population having the same ethnicity and gender as that of the tested individual.

Particularly useful HLA alleles that may be tested in the method of the invention include the HLA DRB3*02 group of alleles. In one embodiment, the method classifies the prognosis of an individual suspected of having breast cancer. In a preferred embodiment, the method classifies the prognosis of an individual suspected of having early onset breast cancer. Classifying the individual as having a poor prognosis can be based upon an analysis of either a single HLA DRB3*02 allele, multiple HLA DRB3*02 alleles, or an HLA DRB3*02 allele and other HLA alleles.

In another aspect, the invention features a method of treating an individual. The method includes introducing into the individual an expression vector coding for expression of an HLA molecule. In a preferred embodiment, the individual is suspected of having cancer. Preferably, the individual is suspected of having breast cancer, more preferably early onset breast cancer. The expression vector may encode HLA DQB1*03032, an HLA DRB1*11 molecule, or any combination of these HLA molecules.

The invention also features a method of treating an individual. The method includes the steps of: (1) determining that the individual does not express a particular HLA allele; and (2) introducing into the individual cells expressing the particular HLA allele. In a preferred embodiment, the individual is suspected of having cancer. Preferably, the individual is suspected of having breast cancer, more preferably early onset breast cancer.

HLA alleles whose absence can be detected in step (1) of the method include HLA DQB1*03032 and the HLA DRB1*11 group of alleles. The method can be used to determine that the individual does not express either one HLA allele or multiple HLA alleles. The cells that are introduced into the individual can express either one HLA allele, e.g., HLA DQB1*03032 or an HLA DRB1*11 allele, or multiple HLA alleles that are not expressed by the individual.

In one embodiment of the method, the cells that are introduced into the individual in step (2) of the method are derived from the individual. In this embodiment, an expression vector is introduced into the cells ex vivo. The individual's cells may be reintroduced to the individual in the context of autologous bone marrow transplantation.

In other embodiments, the cells that are introduced into the individual in step (2) of the method are of allogeneic or xenogeneic origin. Allogeneic or xenogeneic cells can either be derived from an organism that expresses a desired HLA allele, e.g., HLA DQB1*03032 or an HLA DRB1*11 allele, or the allogeneic or xenogeneic cells can be engineered in vitro to express a desired HLA allele.

In preferred embodiments, the individual expresses an HLA DRB3*02 allele. Preferably, the cells that are introduced into the individual in step (2) of the method do not express an HLA DRB3*02 allele.

In one embodiment, the cells that are introduced into the individual in step (2) of the method are dendritic cells. In another embodiment, the cells are hematopoietic stem cells. Preferably, the cells that are introduced into the individual include either cell types that normally express HLA class II molecules or cells that give rise to cell types that normally express HLA class II molecules, e.g., progenitor cells.

Unless otherwise defined, all technical and scientific terms used herein have the same meaning as commonly understood by one of ordinary skill in the art to which this invention belongs. Methods and materials similar or equivalent to those described herein can be used in the practice or testing of the present invention. The present materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description, and from the claims.

### DETAILED DESCRIPTION

The present study provides a detailed molecular analysis of HLA DPB, DQB, and DRB alleles in patients with breast cancer and in ethnically matched controls. This analysis demonstrates the existence of HLA alleles that can confer susceptibility or resistance to breast cancer and potentially to other human cancers as well. Women with early onset breast cancer (diagnosed at or before the age of 40) constitute a subset of the population at increased risk for genetic predisposition to breast cancer (Claus et al. (1991) Am. J. Hum. Genet. 48:232-242) and were therefore chosen for this analysis. The breast cancer-associated HLA alleles identified in this study may be used as diagnostic and prognostic targets as well as in therapeutic and prophylactic methods.

Patients and Controls

One hundred and eighty six consecutive women with breast cancer diagnosed before the age of 40 at hospitals in Boston, Mass., were included in the study (Fitzgerald et al. (1996) N. Eng. J. Med. 334:143-149). Information regarding ethnicity was obtained from each patient. Results from 176 Caucasian patients are included in this report. Two hundred and fifteen healthy Caucasians were included as controls. Ninety-three of these controls have been described earlier (Forcione et al. (1996) Proc. Natl. Acad. Sci. USA 93:5094-5098). The remainder included healthy volunteer blood donors at Massachusetts General Hospital. Information on ethnicity was also obtained from controls.

HLA Class II Typing

Genotyping of DPB1, DQB1, and DRB alleles was performed using a PCR-SSO (sequence-specific oligonucleotide) approach according to the protocols described in the 11th and 12th International HLA Workshops (Tsuji et al.(1991) In HLA 1991: Proceedings of the Eleventh International Histocompatibility Workshop and Conference, Oxford University Press, Oxford. 1:1065-1220; Bignon et al. (1997) In HLA, Genetic Diversity of HLA: Functional and Medical Implications. Proceedings of the Twelfth International Histocompatibility Workshop and Conference EDK, Paris. 1:584-595). SSO probes are used as diagnostic tools to identify polymorphic sequence motifs.

PCR reactions were performed in a total volume of 200 μl and included 3 μl of genomic DNA, 200 pM of each primer, 0.2 mM of each deoxynucleotide triphosphate, 2 mM MgCl₂, 10 mM Tris-HCl pH 8.0, 50 mM KCl, 0.001% (w/v) gelatin and 2.5 U of Taq DNA polymerase (Fisher). Samples were denatured at 96° C. for 6 minutes. Forty cycles of amplification were carried out, each cycle including the following steps: (1) incubation at 96° C. for one minute; (2) annealing for one minute (at variable temperatures, dependent upon the allele being analyzed); and (3) extension at 72° C. for two minutes. Following amplification, samples were incubated at 72° C. for 10 minutes.

The annealing temperatures were: 55° C. for the generic DRB1 alleles; 60° C. for DRB1 and DRB3 group-specific amplifications; 60° C. for DPB1; and 55° C. for DQB1. Ten μl of the reaction was run on a 1.5% agarose gel and the PCR products were viewed under UV light. Controls included previously typed samples. Generic typing of DRB1, DRB3, DRB4 and DRB5 loci was performed using a single pair of generic primers for PCR amplification followed by hybridization of filters with 29 different group-identifying SSOs. For group specific DRB typing, genomic DNA was amplified with five different 5′ primers specific for DRB1-DR1, DRB1-DR2, DRB1-DR4, the DRB1-DR52 associated group and the DRB3-DR52 group. The 3′ primer in the above cases was the same as that used for generic DRB typing. A total of 50 different SSOs were used in the group-specific typing studies. Five SSOs were used for DRB1-DR1 (DRB1*0101-03), 12 for DRB1-DR2 (DRB1*1501-03, DRB1*1601-02), 9 for DRB1-DR4 (DRB1*0401-0411), 12 for the DRB1-DR52 associated group (DRB1*0301/02, DRB1*0801-0804, DRB1*1101-04, DRB1*1201/02, DRB1*1301-05, DRB1*1401-08), and for the DRB3-DR52 group (DRB3*0101, DRB3*0201/0202 and DRB3*0301).

Generic primers were used for DPB1 and DQB1 typing. Twenty five SSOs were used to type 36 DPB1 alleles, and 20 SSOs for 17 DQB1 alleles. Details of the hybridization conditions have been published earlier (Forcione et al. (1996) Proc. Natl. Acad. Sci. USA 93:5094-5098; Cariappa et al. (1998) Gut 43:210-215). Filters were pre-hybridized overnight at 54° C. in a buffer containing 3M tetrarnethylammonium chloride, 50 mM Tris-HCl pH 8.0 and 2 mM EDTA, 5× Denhardts solution, 0.1% sodium dodecyl sulfate, and 100 μg/ml of salmon sperm DNA. Hybridization was carried out using [γ-³²P] ATP 5′ labeled SSOs at 54° C. for 2 hours. The filters were washed twice at room temperature for 15 minutes each in a solution containing 2× SSPE and 0.1% SDS followed by three washes for 10 min at 58° C. in a buffer containing 3M tetramethylammonium chloride, 50 mM Tris-HCl, 2 mM EDTA and 0.1% SDS. Each filter was exposed twice for autoradiography, once for 1-2 hours and subsequently for 14-16 hours. Reactivity was graded visually, using a scale recommended by the 11th International HLA Workshop (Tsuji et al.(1991) In HLA 1991: Proceedings of the Eleventh International Histocompatibility Workshop and Conference, Oxford University Press, Oxford. 1:1065-1220). The use of multiple oligonucleotide probes facilitated definitive identification of negative and positive alleles.

Statistical Analysis

Two tailed uncorrected p values were reported using Fisher's exact test for the analyses of HLA class II allele frequencies. The relative risk was calculated as an odds ratio using the approximation of Woolf (see Woolf(1955) Ann. Hum. Genet. 19:251-253). p values were corrected for the number of comparisons essentially using a modified Bonferroni correction as suggested by Svejgaard and Ryder (see Svejgaard and Ryder (1994) Tissue Antigens 43:19-27). The number of alleles assayed from a given specific PCR amplification reaction was used as the basis for the number of comparisons made. Alleles for which the combined frequency in patients and controls was less than 1 were not included in the number of comparisons. In the case of the DPB1 locus the number of comparisons made was 33. Thus the nominal level for comparison was p≦0.0015. In the case of the DQB1 locus, the number of comparisons made was 16, and the nominal level for comparison was p≦0.0031. In the case of the DRB1 locus, the number of comparisons was 31, and the nominal level for comparison was p≦0.0016. In the case of the DRB3 locus, the number of comparisons was 3, and the nominal level for comparison was p≦0.0166.

DPB1 Alleles

The incidence of DPB1 alleles was compared in 157 breast cancer patients and 207 controls (Table 1). No strong negative or positive association was noted for any DPB1 alleles in patients with breast cancer. A weak negative association was seen for DPB1*0401 (p=0.0042; corrected p=0.126), and a weak positive association was noted for DPB1*3301 (p=0.0061; corrected p=0.183).

TABLE 1
Frequency of DPB1 Alleles in Breast Cancer Patients and Controls

| Allele | Controls | | Breast Cancer | | p Value # |
|---|---|---|---|---|---|
| DPB1*0101 | 8.2% | (17) | 5.1% | (8) | 0.2981 |
| DPB1*0201 | 16.4% | (34) | 22.3% | (35) | 0.1776 |
| DPB1*0202 | 1.4% | (3) | 0.6% | (1) | 0.6371 |
| DPB1*0301 | 17.4% | (36) | 17.2% | (27) | 1.0000 |
| DPB1*0401 | 52.2% | (108) | 36.9% | (58) | 0.0042 |
| DPB1*0402 | 25.6% | (53) | 21.0% | (33) | 0.3218 |
| DPB1*0501 | 1.9% | (4) | 4.5% | (7) | 0.2184 |
| DPB1*0601 | 1.0% | (2) | 4.5% | (7) | 0.0429 |
| DPB1*0801 | 1.9% | (4) | 1.9% | (3) | 1.0000 |
| DPB1*0901 | 0.0% | (0) | 1.3% | (2) | 0.1854 |
| DPB1*1001 | 6.3% | (13) | 1.3% | (2) | 0.0172 |
| DPB1*1101 | 1.9% | (4) | 3.2% | (5) | 0.5077 |
| DPB1*1301 | 4.3% | (9) | 0.6% | (1) | 0.0479 |
| DPB1*1401 | 3.4% | (7) | 2.5% | (4) | 0.7632 |
| DPB1*1501 | 1.4% | (3) | 2.5% | (4) | 0.4704 |
| DPB1*1601 | 1.9% | (4) | 1.3% | (2) | 0.7026 |
| DPB1*1701 | 1.9% | (4) | 3.8% | (6) | 0.3388 |
| DPB1*1801 | 0.5% | (1) | 2.5% | (4) | 0.1700 |
| DPB1*1901 | 0.5% | (1) | 0.6% | (1) | 1.0000 |
| DPB1*2001 | 4.8% | (10) | 5.7% | (9) | 0.8130 |
| DPB1*2201 | 0.0% | (0) | 0.6% | (1) | 0.4313 |
| DPB1*2301 | 23.7% | (49) | 19.1% | (30) | 0.3077 |
| DPB1*2401 | 1.9% | (4) | 1.9% | (3) | 1.0000 |
| DPB1*2501 | 1.4% | (3) | 5.7% | (9) | 0.0350 |
| DPB1*2601 | 1.0% | (2) | 3.2% | (5) | 0.1459 |
| DPB1*2701 | 2.9% | (6) | 1.9% | (3) | 0.7374 |
| DPB1*2801 | 0.0% | (0) | 0.6% | (1) | 0.4313 |
| DPB1*2901 | 1.4% | (3) | 5.1% | (8) | 0.0620 |
| DPB1*3101 | 1.9% | (4) | 0.0% | (0) | 0.1371 |
| DPB1*3201 | 1.9% | (4) | 7.0% | (11) | 0.0297 |
| DPB1*3301 | 0.5% | (1) | 5.1% | (8) | 0.0061 |
| DPB1*3401 | 0.0% | (0) | 0.6% | (1) | 0.4313 |
| DPB1*3501 | 1.4% | (3) | 1.3% | (2) | 1.0000 |

#Uncorrected two-tailed p value (Fisher's Exact Test)

DQB1 Alleles

The incidence of DQB1 alleles was compared in 176 breast cancer patients and 199 controls (Table 2). The DQB*03032 allele was found in 14 controls and in 0 patients with breast cancer (p=0.0001) (see Table 2). The Relative Risk was 0.0358. The corrected p value for this negative association is 0.0016, which is highly significant.

TABLE 2
Frequency of DQB1 Alleles in Breast Cancer Patients and Controls

| Allele | Controls | | Breast Cancer | | p Value # |
|---|---|---|---|---|---|
| DQB1*0201 | 30.7% | (61) | 39.8% | (70) | 0.0663 |
| DQB1*0301 | 40.2% | (80) | 36.4% | (64) | 0.4584 |
| DQB1*0302 | 28.1% | (56) | 20.5% | (36) | 0.0930 |
| DQB1*03031 | 1.0% | (2) | 0.6% | (1) | 1.0000 |
| DQB1*03032 | 7.0% | (14) | 0.0% | (0) | 0.0001** |
| DQB1*0401 | 0.5% | (1) | 3.4% | (6) | 0.0545 |
| DQB1*0402 | 4.5% | (9) | 1.1% | (2) | 0.0670 |
| DQB1*0501 | 17.1% | (34) | 21.6% | (38) | 0.2946 |
| DQB1*0502 | 3.5% | (7) | 4.0% | (7) | 1.0000 |
| DQB1*05031 | 6.5% | (13) | 3.4% | (6) | 0.2381 |
| DQB1*05032 | 0.5% | (1) | 0.0% | (0) | 1.0000 |
| DQB1*0504 | 0.0% | (0) | 0.0% | (0) | — |
| DQB1*0601 | 2.0% | (4) | 5.7% | (10) | 0.0986 |
| DQB1*0602 | 26.6% | (53) | 22.7% | (40) | 0.4035 |
| DQB1*0603 | 10.0% | (20) | 10.2% | (18) | 1.0000 |
| DQB1*0604 | 4.5% | (9) | 3.4% | (6) | 0.6105 |
| DQB1*0605 | 1.0% | (2) | 4.5% | (8) | 0.0506 |

#Uncorrected two-tailed p value (Fisher's Exact Test)
**Significant after correction; nominal value for comparison p ≦ 0.0031

DRB1 Alleles

The incidence of DRB1 alleles was compared in 173 breast cancer patients and 215 controls (Table 3). The DRB1*11 group of alleles was found in 35 controls, but in only 6 patients with breast cancer (p<0.0001). The Relative Risk was 0.1846. The corrected p value for this negative association is <0.0030, which is highly significant.

At least 27 different DRB1*11 alleles have been described in recent years, many of which remain to be confirmed. The frequencies with which many of these recently identified alleles occur in Caucasians has not been established.

TABLE 3
Frequency of DRB1 Alleles in Breast Cancer Patients and Controls

| Allele | Controls | | Breast Cancer | | p Value # |
|---|---|---|---|---|---|
| DRB1*0101 | 11.6% | (25) | 13.3% | (23) | 0.6442 |
| DRB1*0102 | 4.2% | (9) | 5.8% | (10) | 0.4875 |
| DRB1*0103 | 2.8% | (6) | 5.8% | (10) | 0.1984 |
| DRB1*1501 | 27.9% | (60) | 20.2% | (35) | 0.0963 |
| DRB1*1502 | 0.9% | (2) | 1.7% | (3) | 0.6597 |
| DRB1*1503 | 0.9% | (2) | 0.6% | (1) | 1.0000 |
| DRB1*1601 | 2.8% | (6) | 2.9% | (5) | 1.0000 |
| DRB1*1602 | 1.4% | (3) | 1.2% | (2) | 1.0000 |
| DRB1*0301-02 | 20.5% | (44) | 30.1% | (52) | 0.0333 |
| DRB1*0401 | 6.5% | (14) | 4.0% | (7) | 0.3684 |
| DRB1*0402 | 5.6% | (12) | 4.6% | (8) | 0.8183 |
| DRB1*0403 | 1.4% | (3) | 0.0% | (0) | 0.2569 |
| DRB1*0404 | 10.7% | (23) | 11.6% | (20) | 0.8711 |
| DRB1*0405 | 1.9% | (4) | 0.6% | (1) | 0.3865 |
| DRB1*0407 | 2.3% | (5) | 1.2% | (2) | 0.4682 |
| DRB1*0408 | 7.4% | (16) | 6.4% | (11) | 0.8413 |
| DRB1*0409 | 0.0% | (0) | 0.0% | (0) | — |
| DRB1*0410 | 0.0% | (0) | 0.0% | (0) | — |
| DRB1*0411 | 0.0% | (0) | 0.0% | (0) | — |
| DRB1*0701 | 19.5% | (42) | 24.9% | (43) | 0.2189 |
| DRB1*0801-04 | 4.2% | (9) | 6.4% | (11) | 0.3632 |
| DRB1*0901A/B | 2.8% | (6) | 0.6% | (1) | 0.1370 |
| DRB1*1001 | 0.9% | (2) | 1.2% | (2) | 1.0000 |
| DRB1*1101-04 | 16.3% | (35) | 3.5% | (6) | <0.0001** |
| DRB1*1201-02 | 3.2% | (7) | 5.2% | (9) | 0.4425 |
| DRB1*1301 | 5.1% | (11) | 0.6% | (1) | 0.0147 |
| DRB1*1302 | 10.2% | (22) | 12.1% | (21) | 0.6263 |
| DRB1*1303 | 0.9% | (2) | 4.6% | (8) | 0.0268 |
| DRB1*1304 | 0.0% | (0) | 0.0% | (0) | — |
| DRB1*1305 | 0.0% | (0) | 0.6% | (1) | 0.4459 |
| DRB1*1401 | 0.0% | (0) | 1.2% | (2) | 0.0441 |
| DRB1*1402 | 6.5% | (14) | 0.6% | (1) | 0.4459 |
| DRB1*1403 | 6.5% | (14) | 7.5% | (13) | 0.6944 |
| DRB1*1404 | 1.9% | (4) | 1.2% | (2) | 0.6961 |
| DRB1*1405 | 2.8% | (6) | 5.8% | (10) | 0.1984 |
| DRB1*1406 | 0.0% | (0) | 0.0% | (0) | — |
| DRB1*1407 | 0.0% | (0) | 0.0% | (0) | — |
| DRB1*1408 | 0.0% | (0) | 0.0% | (0) | — |

#Uncorrected two-tailed p value (Fisher's Exact Test)
**Significant after correction; nominal value for comparison p ≦ 0.0016

DRB1*11 alleles are not in linkage disequilibrium with DQB*03032. In Caucasians DQB*03032 is in very weak linkage disequilibrium with DRB1*0701, DRB1*0901, and DRB1*1602. It is clear from Table 3 thatthe negative association of DQB*03032 and breast cancer does not represent linkage disequilibrium with a known DRB1 gene.

The negative associations described herein suggest that mammary tumor-specific peptides may lodge in the antigen binding grooves of specific HLA class II heterodimers in resistant individuals. Peptides bound to DQB*03032 and DRB1*11 may be presented to T cells in resistant individuals.

DRB3 Alleles

The incidence of DRB3 alleles was compared in 171 breast cancer patients and 208 controls (Table 4). Over half of the patients with breast cancer (94 out of a total of 171, 55%) and a substantial but smaller proportion of the controls (85 of 208, 40.9%) inherited a DRB3*02 allele (see Table 4). The p value for this positive association is 0.0072. The corrected p value is 0.0216, which is significant.

TABLE 4
Frequency of DRB3 Alleles in Breast Cancer Patients and Controls

| Allele | Controls | Breast Cancer | p Value # |
|---|---|---|---|
| DRB3*0101 | 24.5% (51) | 25.1% (43) | 0.9053 |
| DRB3*0201/*0202 | 40.9% (85) | 55.0% (94) | 0.0072** |
| DRB3*0301 | 7.7% (16) | 9.4% (16) | 0.5823 |

#Uncorrected two-tailed p value (Fisher's Exact Test)
**Significant after correction; nominal value for comparison p ≦ 0.0166

A significant positive association was noted with DRB3*02 alleles and breast cancer. The positive association of a specific HLA class II allele in a cancer may reflect the role pf a specific HLA class II molecule either in promoting chronic inflammation or in influencing the development of a hole in the T cell repertoire, e.g., during thymic education. Although lymphocytic infiltration and fibrosis are frequently seen in human breast cancer, there is little clinical evidence to suggest that breast cancer in women develops in a setting of chronic inflammation. In an individual, CD4⁺CD8⁺ double positive thymocytes bearing T cell receptors capable of avidly recognizing self-MHC molecules are eliminated during thymic education. The presentation of self peptides by a breast cancer-associated HLA class II allele may eliminate certain T cell clones that have the potential to respond to specific tumor antigens.

The patient group in this study contained 17 Jewish subjects and 13 Jewish controls. When the data was separately analyzed, excluding Jewish patients and controls, the negative associations of DQB1*03032 and the DRB1*11 alleles in breast cancer remained highly significant (DQB1*03032 p=0.0002; DRB1*11 p=0.0006). The numbers of Jewish patients and controls was insufficient for this sub-group to be analyzed separately in a statistically meaningful manner. None of the Jewish patients or controls inherited DQB1*03032. Five out of 13 Jewish controls (38.5%) and 0 out of 17 Jewish patients (0%) inherited a DRB1*11 allele (p=0.0090).

Susceptibility and Prognosis Determinations

The associations described herein of particular HLA alleles with human cancer can be exploited to evaluate an individual for susceptibility to cancer, e.g., breast cancer, particularly early onset breast cancer. Susceptibility to other cancers may also be evaluated using methods of the invention, e.g., cancer of the lung, thyroid, hematopoietic system, gastrointestinal tract, genito-urinary tract, colon, kidney, prostate, small intestine, esophagus, and pancreas. Additionally, these associations may be used to determine the prognosis of an individual suspected of having cancer. In either case, the individual may be typed at one or more relevant HLA loci, e.g., DQB1, DRB1, and/or DRB3. Following the HLA typing, an evaluation can be made of the individual's susceptibility or prognosis. HLA typing can be performed by standard techniques, e.g., the typing techniques described herein. In general, the presence of an aliele associated with resistance to cancer is indicative of a decreased or low susceptibility or a positive prognosis, whereas the absence of the allele predicts an increased susceptibility or a poor prognosis. Alternatively, the presence of an allele positively associated with cancer is indicative of anincreased susceptibility or a poor prognosis, whereas the absence of that allele predicts a decreased or low susceptibility or a positive prognosis. These HLA-based predictions may be used, at least in part, to determine the appropriate treatment protocol for the tested individual.

Because specific HLA alleles are associated with resistance to breast cancer, e.g., DQB1*03032 and DRB1*11 alleles, the presence of one or more of these alleles in an individual can be used as an indicator of a decreased or low susceptibility to breast cancer. For example, an individual can be evaluated for susceptibility to cancer by determining whether the individual bears an DQB1*03032 and/or DRB1*11 allele, and, if the individual does bear one or more of these alleles, identifying the individual as having a decreased or low susceptibility to breast cancer. Additionally, the presence of one or more of the DQB1*03032 or DRB1*11 alleles can be used as an indicator of a positive prognosis in an individual suspected of having breast cancer. Alternatively, the absence in an individual of one or more alleles associated with resistance to breast cancer, e.g., DQB1*03032 and DRB1*11 alleles, can be used as an indicator of an increased or average susceptibility to breast cancer or an indicator of poor or average prognosis in an individual suspected of having the disease.

HLA alleles positively associated with breast cancer, e.g. DRB3*02 alleles, can also be used as an indicator of susceptibility and/or prognosis. For example, the presence of a DPRB3*02 allele can be used as an indicator of an increased susceptibility to breast cancer. Additionally, the presence of this allele can be used as an indicator of a poor prognosis in an individual suspected of having breast cancer. Alternatively, the absence in an individual of an allele positively associated with breast cancer, e.g., a DRB3*02 allele, can be used as an indicator of an decreased or average susceptibility to breast cancer or an indicator of positive or average prognosis in an individual suspected of having the disease.

Therapeutic Treatment

An individual suspected of having cancer, e.g., breast cancer, or of being susceptible to developing cancer can be treated by supplying a HLA therapeutic composition to the individual. Various cancers may be treated using a HLA therapeutic composition of the invention, e.g., cancer of the breast, lung, thyroid, hematopoietic system, gastrointestinal tract, genito-urinary tract, colon, kidney, prostate, small intestine, esophagus, and pancreas.

HLA therapeutic compositions include a nucleic acid coding for a functional HLA protein, e g. a DQB1*03032 or DRB1*11 polypeptide. A nucleic acid (e.g., a cDNA) encoding the HLA molecule is operably linked to expression control elements (e.g., promoter and enhancer) that induce expression in desired tissues, e.g., a hematopoietic stem cell or an APC. The nucleic acid may be incorporated into a vector appropriate for transforming the cells, such as a plasmid, retrovirus, adenovirus, or adeno-associated virus. One of the many other known types of techniques for introducing DNA into cells in vivo may be used (e.g., liposomes or delivery of naked DNA). The HLA therapeutic composition can be administered to an individual who either fails to express a particular HLA protein or expresses an inadequate amount of the protein.

Receptor-mediated targeted delivery of therapeutic compositions containing HLA nucleic acids to specific tissues can also be used. Receptor-mediated DNA delivery techniques are described in, for example, Findeis et al. (1993), Trends in Biotechnol. 11, 202-05; Chiou et al. (1994), Gene Therapeutics: Methods and Applications of Direct Gene Transfer (J. A. Wolff, ed.); Wu & Wu (1988), J. Biol. Chem. 263, 621-24; Wu et al. (1994), J. Biol. Chem. 269, 542-46; Zenke et al. (1990), Proc. Natl. Acad. Sci. U.S.A. 87, 3655-59; Wu et al. (1991), J. Biol. Chem. 266, 338-42.

Alternatively, an HLA therapeutic composition can be introduced into an individual's cells, e.g., dendritic cells or hematopoietic stem cells, ex vivo, and the cells can then implanted into the individual. For example, the HLA therapeutic composition can be delivered to an individual's cells in the context of autologous bone marrow transplantation. Cells can be removed from a variety of locations, such as hernatopoietic tissues, e.g., bone marrow or peripheral blood. The removed cells can then be contacted with the HLA therapeutic composition utilizing any of the above-described techniques, followed by the return of the cells to the human. When introducing the HLA therapeutic composition to a population of cells, the composition may either be delivered to the entire population of isolated cells, or the cells can be enriched for particularly useful cell types, e.g., hematopoietic stem cells or APCs such as dendritic cells, prior or subsequent to the delivery of the HLA therapeutic composition.

Both the dose of the HLA composition and the means of administration can be determined based on the specific qualities of the therapeutic composition, the condition, age, and weight of the patient, the progression of the disease, and other relevant factors.

Individuals suspected of having breast cancer or suspected of being susceptible to developing breast cancer can also be treated by allogeneic or xenogeneic cell transplantation, e.g., bone marrow transplantation. For example, cell transplantation can be performed on an individual that expresses an HLA allele that is positively associated with breast cancer, e.g. DR3*02. The donor cells can possess any HLA genotype, so long as the cells do not express the allele positively associated with breast cancer. Preferred donor cells would bear one or more HLA alleles associated with resistance to breast cancer, either naturally or as a result of genetic manipulation. In another example, an individual lacking an HLA allele associated with resistance to breast cancer can be transplanted with cells from an allogeneic donor that express a particular resistance allele, e.g., DQB1*03032 or DRB1*11. In the case of xenogeneic transplantation, the donor cells can be engineered to express an appropriate HLA molecule. The donor cells can either be engineered in vitro or they can be derived from an organism such as a transgenic pig, cow, horse or other mammal that expresses a human HLA molecule. Because certain HLA alleles, e.g. DRB3*02, are positively associated with human cancer, the donor cells preferably do not express that particular allele.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of evaluating an individual's susceptibility to cancer, the method comprising:
determining that the individual bears an HLA DQB1*03032 allele, and
classifying the individual as having a low susceptibility to cancer based upon the presence of the HLA DQB1*03032 allele.

2. The method of claim 1, wherein the cancer is breast cancer.

3. The method of claim 2, wherein the cancer is early onset breast cancer.

4. The method of claim 1, further comprising:
determining that the individual bears an HLA DRB1*11 allele, and
classifying the individual as having a low susceptibility to cancer based upon the presence of both the HLA DQB1*03032 allele and an HLA DRB1*11 allele.

5. A method of evaluating an individual's susceptibility to cancer, the method comprising:
determining that the individual bears an HLA DRB1*11 allele, and
classifying the individual as having a low susceptibility to cancer based upon the presence of the HLA DRB1*11 allele.

6. The method of claim 5, wherein the cancer is breast cancer.

7. The method of claim 6, wherein the cancer is early onset breast cancer.

8. A method of evaluating an individual's susceptibility to cancer, the method comprising:
determining that the individual bears an HLA DRB3*02 allele, and
classifying the individual as having a high susceptibility to cancer based upon the presence of the HLA DRB3*02 allele.

9. The method of claim 8, wherein the cancer is breast cancer.

10. The method of claim 9, wherein the cancer is early onset breast cancer.

11. A method of evaluating the prognosis of an individual suspected of having cancer, the method comprising:
determining that the individual bears an HLA DQB1*03032 allele, and
classifying the individual as having a positive prognosis based upon the presence of the HLA DQB1*03032 allele.

12. The method of claim 11, wherein the cancer is breast cancer.

13. The method of claim 12, wherein the cancer is early onset breast cancer.

14. The method of claim 11, further comprising:
determining that the individual bears an HLA DRB1*11 allele, and
classifying the individual as having a positive prognosis based upon the presence of both the HLA DQB1*03032 allele and an HLA DRB1*11 allele.

15. A method of evaluating the prognosis of an individual suspected of having cancer, the method comprising:
determining that the individual bears an HLA DRB1*11 allele, and
classifying the individual as having a positive prognosis based upon the presence of an HLA DRB1*11 allele.

16. The method of claim 15, wherein the cancer is breast cancer.

17. The method of claim 16, wherein the cancer is early onset breast cancer.

18. A method of evaluating the prognosis of an individual suspected of having cancer, the method comprising:
determining that the individual bears an HLA DRB3*02 allele, and
classifying the individual as having a poor prognosis based upon the presence of the HLA DRB3*02 allele.

19. The method of claim 18, wherein the cancer is breast cancer.

20. The method of claim 19, wherein the cancer is early onset breast cancer.

21. A method of treating an individual, the method comprising introducing into the individual an expression vector coding for expression of HLA DQB1*03032.

22. The method of claim 21, wherein the individual is suspected of having cancer.

23. The method of claim 22, wherein the cancer is breast cancer.

24. The method of claim 23, wherein the cancer is early onset breast cancer.

25. The method of claim 24, wherein the expression vector further codes for expression of an HLA DRB1*11 molecule.

26. A method of treating an individual, the method comprising introducing into the individual an expression vector coding for expression of an HLA DRB1*11 molecule.

27. The method of claim 26, wherein the individual is suspected of having cancer.

28. The method of claim 27, wherein the cancer is breast cancer.

29. The method of claim 28, wherein the cancer is early onset breast cancer.

30. A method of treating an individual, the method comprising
determining that the individual does not express HLA DQB1*03032, and
introducing into the individual cells expressing HLA DQB1*03032.

31. The method of claim 30, wherein the individual is suspected of having cancer.

32. The method of claim 31, wherein the cancer is breast cancer.

33. The method of claim 32, wherein the cancer is early onset breast cancer.

34. The method of claim 30, wherein the cells are derived from the individual and an expression vector is introduced into the cells ex vivo.

35. The method of claim 34, wherein the cells are dendritic cells.

36. The method of claim 34, wherein the cells are hematopoietic stem cells.

37. The method of claim 30, wherein the individual expresses an HLA DRB3*02 allele.

38. The method of claim 30, wherein the cells are of allogeneic origin.

39. The method of claim 30, wherein the cells are of xenogeneic origin.

40. A method of treating an individual, the method comprising
determining that the individual does not express an HLA DRB1*11 allele, and
introducing into the individual cells expressing an HLA DRB1*11 allele.

41. The method of claim 40, wherein the individual is suspected of having cancer.

42. The method of claim 41, wherein the cancer is breast cancer.

43. The method of claim 42, wherein the cancer is early onset breast cancer.

44. The method of claim 40, wherein the cells are derived from the individual and an expression vector is introduced into the cells ex vivo.

45. The method of claim 44, wherein the cells are dendritic cells.

46. The method of claim 44, wherein the cells are hematopoietic stem cells.

47. The method of claim 40, wherein the individual expresses an HLA DRB3*02 allele.

48. The method of claim 40, wherein the cells are of allogeneic origin.

49. The method of claim 40, wherein the cells are of xenogeneic origin.
